(19) United States
(12) Patent Application Publication
(10) Pub. No.: US 2025/0242095 A1
(43) Pub. Date: Jul. 31, 2025

(54) STORAGE DEVICE OF BREAST PUMP

(71) Applicant: Shenzhen Oceanwing Smart Innovations Technology Co., Ltd, Shenzhen (CN)

(72) Inventors: Shanshan Xiao, Shenzhen (CN); Zhihai Zhang, Shenzhen (CN); Liying Chen, Shenzhen (CN); Yingying Qiao, Shenzhen (CN)

(21) Appl. No.: 19/018,658

(22) Filed: Jan. 13, 2025

(30) Foreign Application Priority Data
Jan. 29, 2024 (CN) 202420217001.0

(51) Int. Cl. A61M 1/06; H02J 7/00

(52) U.S. Cl. CPC A61M 1/062; A61M 2205/8256; A61M 2209/06; H02J 7/0044

(57) ABSTRACT

Disclosed is a storage device of a breast pump, wherein the storage device comprises a storage body and a charging connector. The storage body comprises a storage cavity configured to accommodate the breast pump. The charging connector is movably disposed on the storage body, and configured to charge the breast pump. The charging connector is movable relative to the storage body to adjust a position of the charging connector based on a position of the breast pump in the storage cavity. The storage device comprises a fixing bracket fixedly disposed on the storage body. The fixing bracket and the charging connector are movably connected so that the charging connector is movable relative to the storage body.

### CROSS-REFERENCE TO RELATED APPLICATION

The present application claims priority to Chinese Patent Application No. 202420217001.0, titled “Storage Device of Breast Pump,” filed on Jan. 29, 2024. The above applications is incorporated herein by reference in its entirety.

### FIELD

The present disclosure relates to the technical field of breast pumps, in particular to a storage device of a breast pump.

### BACKGROUND

With the development of science and technology and people's pursuit of quality of life, various humanized products begin to appear to meet users' living needs considerately. The appearance of breast pumps makes breastfeeding more convenient. A breast pump is used to suck breast milk and store the sucked breast milk in a milk bin assembly, so that it is convenient for an infant to eat the breast milk when needed.

In order to facilitate management of a breast pump, a storage device can be used to supply power to the breast pump while storing the breast pump. At present, due to different sizes and structures of breast pumps, there is a problem of poor compatibility when the storage device is in charging connection with different breast pumps, which affects versatility of the storage device.

### SUMMARY

The technical problem mainly solved by the present disclosure is to provide a storage device of a breast pump, which can improve the compatibility of the storage device with different breast pumps during charging.

In order to solve the technical problems, technical solutions adopted by the disclosure are as follows. Provided is a storage device of a breast pump. The storage device comprises a storage body and a charging connector. The storage body comprises a storage cavity configured to accommodate the breast pump. The charging connector may be movably disposed on the storage body, exposed relative to the storage cavity, and configured to charge the breast pump. The charging connector may be movable relative to the storage body to adjust a position of the charging connector so as to be electrically connected with the breast pump according to a placement position of the breast pump in the storage cavity when the breast pump is placed in the storage cavity. The storage device comprises a fixing bracket fixedly disposed on the storage body. The fixing bracket and the charging connector may be movably connected so that the charging connector is movable relative to the storage body.

Beneficial effects of the disclosure are: different from the related art, the storage body is provided with a storage cavity and configured to accommodate the breast pump, the charging connector is movably disposed on the storage body, exposed relative to the storage cavity, and configured to charge the breast pump, the charging connector is movable relative to the storage body to adjust the position of the charging connector so as to be electrically connected with the breast pump according to a placement position of the breast pump in the storage cavity when the breast pump is placed in the storage cavity. The storage device includes a fixing bracket fixedly disposed on the storage body, and the fixing bracket and the charging connector are movably connected so that the charging connector is movable relative to the storage body. By setting the charging connector to be movable relative to the storage body to adjust the position of the charging connector, the charging connector can be matched and connected with different breast pumps when the storage device stores breast pumps of different structures and sizes, thereby improving the compatibility of the storage device with the breast pumps of different structures and sizes during charging, so that the storage device has a wider range of application scenarios.

### BRIEF DESCRIPTION OF THE DRAWINGS

FIG. 1 is a perspective structure diagram of a storage device of a breast pump according to an example of the present disclosure;

FIG. 2 is an exploded structure diagram of the storage device of a breast pump;

FIG. 3 is a cross-sectional diagram of a connection structure between the breast pump and the storage device;

FIG. 4 is another cross-sectional diagram of a connection structure between the breast pump and the storage device;

FIG. 5 is an enlarged diagram of a part A in FIG. 3;

FIG. 6 is an enlarged diagram of a part B in FIG. 4;

FIG. 7 is a diagram of a connection structure between a charging connector and a fixing bracket shown in FIG. 3;

FIG. 8 is a diagram of a connection structure between a charging connector and a fixing bracket shown in FIG. 4;

FIG. 9 is an exploded structure diagram of the connection structure between the charging connector and the fixing bracket shown in FIG. 8;

FIG. 10 is a mounting structure diagram of a fixing bracket;

FIG. 11 is another exploded structure diagram of a storage device of a breast pump according to an example of the present disclosure; and

FIG. 12 is a structure diagram of the charging connector and the fixing bracket shown in FIG. 11.

### DETAILED DESCRIPTION

Technical solutions in the present disclosure will be clearly and fully described with reference to the accompanying drawings in the present disclosure. Obviously, the features to be described are merely part of examples of the disclosure, but not all examples of the disclosure. Based on the examples of the disclosure, all other examples obtained by those of ordinary skill in the art without inventive work shall fall within the scope of the disclosure.

Through long-term research, the inventor found that in order to facilitate management of the breast pump, a storage device can be configured to supply power to the breast pump while storing the breast pump. At present, due to different sizes and structures of breast pumps, there is a problem of poor compatibility when the storage device is in charging connection with different breast pumps, which affects versatility of the storage device. In order to solve the problem, the present disclosure provides the following examples.

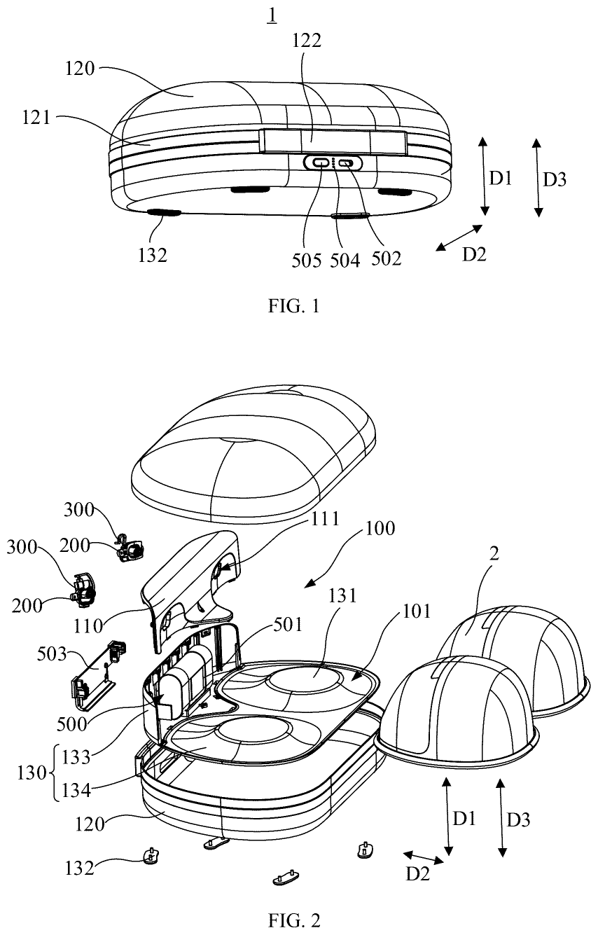

As shown in FIGS. 1 and 2, a storage device 1 according to an example of a storage device of a breast pump of the present disclosure comprises a storage body 100 and a charging connector 200. The storage body 100 is provided with a storage cavity 101 and configured to accommodate the breast pump 2. The charging connector 200 is movably disposed on the storage body 100, exposed relative to the storage cavity 101. A power source is configured to charge the breast pump 2 via the charging connector 200. The charging connector 200 is movable relative to the storage body 100 to adjust the position of the charging connector 200 so as to be electrically connected with the breast pump 2 according to a placement position of the breast pump 2 in the storage cavity 101 when the breast pump 2 is placed in the storage cavity 101.

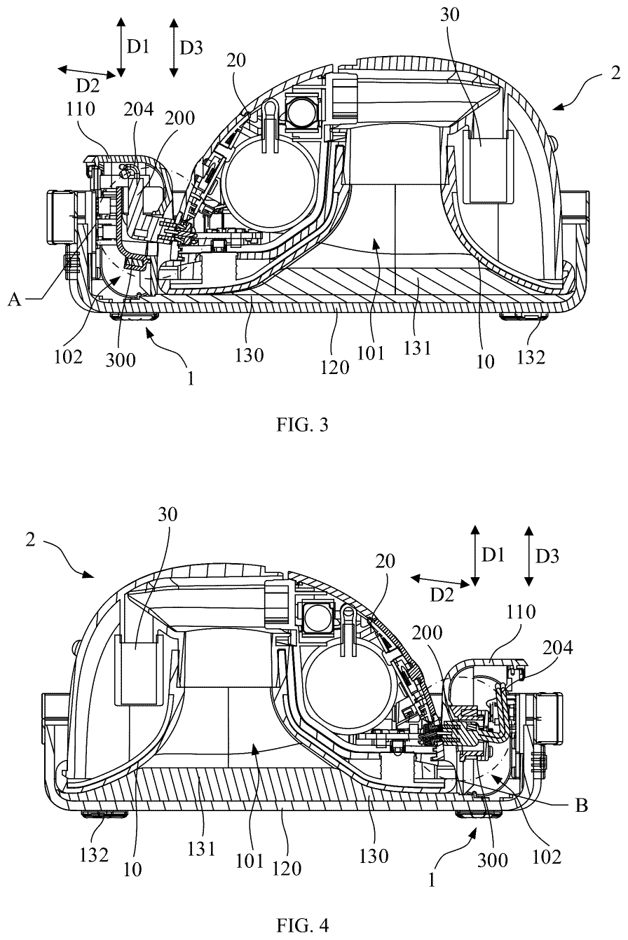

The breast pump 2 can be configured to suck breast milk. Specifically, as shown in FIGS. 3 and 4, the breast pump 2 comprises a detachably connected outer cover assembly 10, a main engine 20, and a milk bin assembly 30. The breast pump 2 is configured to suck breast milk and store the sucked breast milk in the milk bin assembly 30, so that the infant can eat the breast milk when needed. During use of the breast pump 2, a side of the outer cover assembly 10 may be fitted to the human skin, so that breast milk passes through the outer cover assembly 10 into the milk bin assembly 30 located on the other side of the outer cover assembly 10. A suction device may be provided on the main engine 20 located on the other side of the outer cover assembly 10, and when the suction device operates, negative pressure is generated in the outer cover assembly 10 so as to suck breast milk into the milk bin assembly 30. After the breast pump 2 completes operation, the breast pump 2 can be detached into the outer cover assembly 10, the milk bin assembly 30 and the main engine 20 independent from each other, thereby facilitating cleaning of the outer cover assembly 10 and the milk bin assembly 30.

The outer cover assembly 10 may have a heating member (e.g., a heater device such electric pads). The heating member is configured to heat a housing, thereby promoting discharge of milk from the human body, and also improving comfort of a user wearing the breast pump 2. For example, the heating member is an FPC (a flexible printed circuit board). Further, the main engine 20 can supply power to the heating member.

When the breast pump 2 is idle or when the breast pump 2 needs to be carried out, the breast pump 2 can be placed in the storage cavity 101, so that the storage body 100 can protect the breast pump 2. When the breast pump 2 is placed in the storage cavity 101, the breast pump 2 can be charged via the charging connector 200, thereby increasing use time of the breast pump 2 and providing convenience for the user.

By setting the charging connector 200 to be movable relative to the storage body 100 to adjust the position of the charging connector 200, the charging connector 200 can be matched and connected with different breast pumps 2 when the storage device 1 stores breast pumps 2 of different structures and sizes, thereby improving the compatibility of the storage device 1 with the breast pumps 2 of different structures and sizes during charging, so that the storage device 1 has a wider range of application scenarios.

For example, the breast pump 2 is provided with an electrical connection assembly, and the breast pump 2 is electrically connected with the charging connector 200 by the electrical connection assembly. The charging connector 200 is movable relative to the storage body 100 in a height direction D1 of the storage device 1, so that the charging connector 200 can be matched and connected with the electrical connection assemblies of different heights when the storage device 1 stores the breast pump 2 having the electrical connection assemblies of different heights. For another example, the charging connector 200 is movable relative to the storage body 100 in a width direction of the storage device 1 or a length direction of the storage device 1, so that the charging connector 200 can be matched and connected to electrical connection assemblies at different positions in the width direction of the storage device 1 or the length direction of the storage device 1.

The storage body 100 is provided with a storage cavity 101 which is configured to accommodate the breast pump 2. The charging connector 200 is movably disposed on the storage body 100, exposed relative to the storage cavity 101, and configured to charge the breast pump 2. The charging connector 200 is movable relative to the storage body 100 to adjust the position of the charging connector 200 so as to be electrically connected with the breast pump 2 according to the placement position of the breast pump 2 in the storage cavity 101 when the breast pump 2 is placed in the storage cavity 101. By setting the charging connector 200 to be movable relative to the storage body 100 to adjust the position of the charging connector 200, the charging connector 200 can be matched and connected with different breast pumps 2 when the storage device 1 stores breast pumps 2 of different structures and sizes, thereby improving the compatibility of the storage device 1 with the breast pumps 2 of different structures and sizes during charging, so that the storage device 1 has a wider range of application scenarios.

As shown in FIGS. 3 and 4, the storage device 1 comprises a fixing bracket 300 fixedly disposed on the storage body 100. The fixing bracket 300 and the charging connector 200 are movably connected so that the charging connector 200 can move relative to the storage body 100.

By providing the fixing bracket 300, the charging connector 200 is movably disposed on the storage body 100. The fixing bracket 300 may have a limiting effect, thereby limiting the charging connector 200 from being disengaged from the storage body 100.

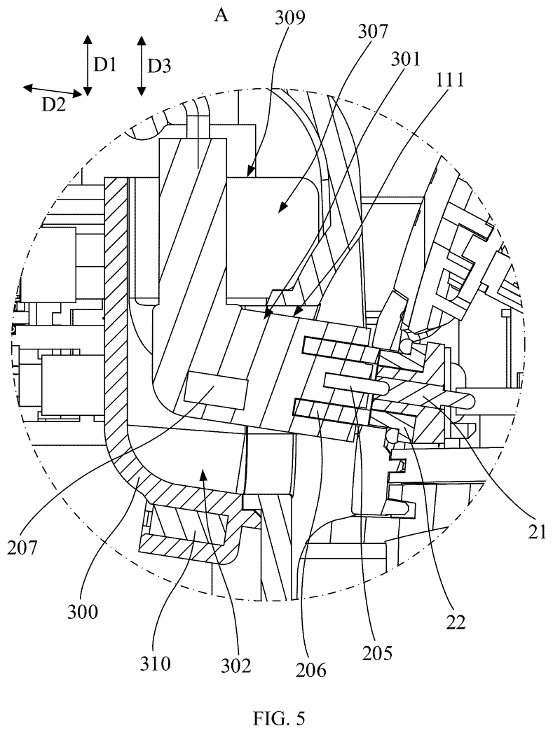

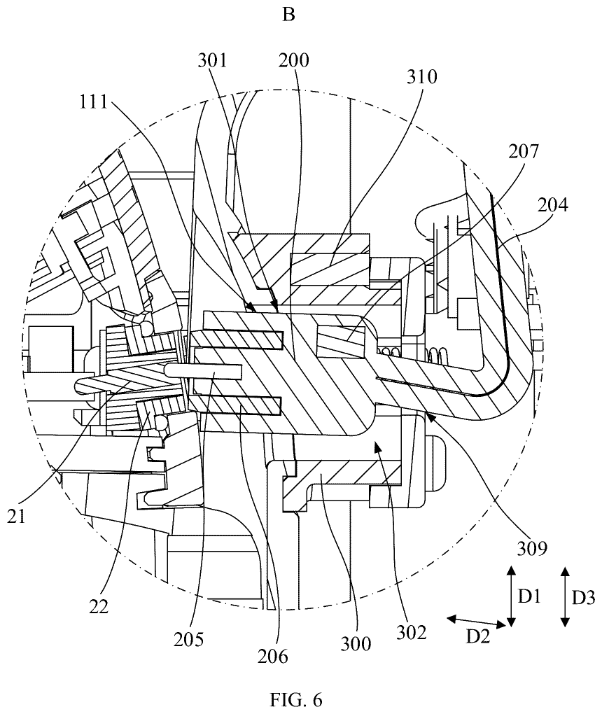

As shown in FIGS. 5-7, the fixing bracket 300 may be provided with an accommodating space 302 having a first opening 301, and at least part of the charging connector 200 is movably accommodated in the accommodating space 302 and movably passes through the first opening 301, and is exposed relative to the storage cavity 101 through the first opening 301.

For example, the charging connector 200 may move in a direction from the first opening 301 toward the storage cavity 101. For another example, the direction from the first opening 301 toward the storage cavity 101 is defined as a preset direction, and the charging connector 200 may move along the length direction or the width direction of the first opening 301 perpendicular to the preset direction.

The accommodating space 302 may communicate with the storage cavity 101 through the first opening 301. In some examples, the charging connector 200 may be in the accommodating space 302 when idle, and the charging connector 200 may partially extend from the accommodating space 302 into the storage cavity 101 through the first opening 301 when charging the breast pump 2, thereby facilitating electrical connection with the breast pump 2. In other examples, the charging connector 200 may be partially in the accommodating space 302 when idle, and the charging connector 200 partially extends into the storage cavity 101 through the first opening 301. This can improve flexibility of a position of the charging connector 200 relative to the breast pump 2 and facilitate matching connection of the charging connector 200 with the breast pump 2.

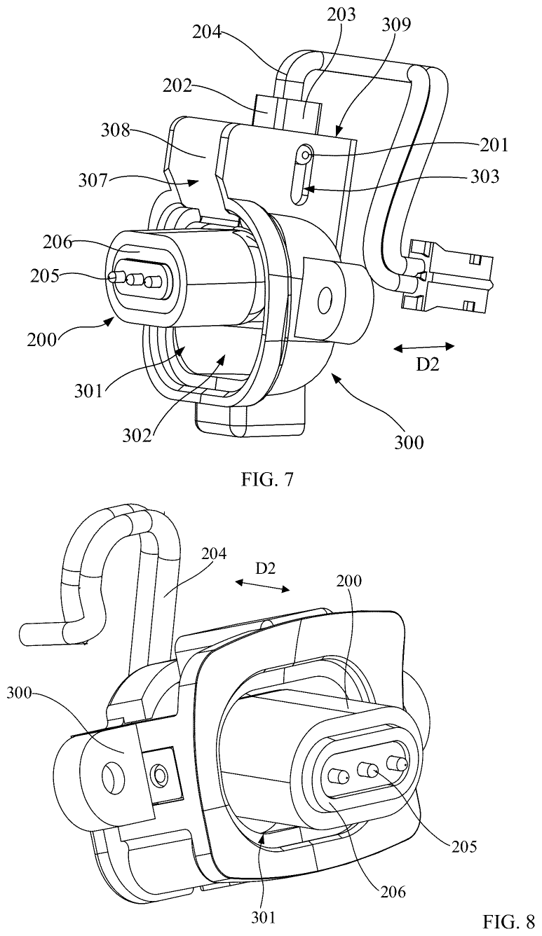

As shown in FIGS. 7 and 8, the charging connector 200 and the fixing bracket 300 are rotatably connected, and/or the charging connector 200 and the fixing bracket 300 can relatively move along the preset direction to adjust the position of the charging connector 200.

Since the charging connector 200 and the fixing bracket 300 are rotatably connected, the charging connector 200 can adjust its own position by rotating relative to the fixing bracket 300, thereby facilitating the matching connection of the charging connector 200 with the breast pump 2.

Since the charging connector 200 and the fixing bracket 300 relatively move along the preset direction, the charging connector 200 can adjust its own position by moving along the preset direction relative to the fixing bracket 300, thereby facilitating the matching connection of the charging connector 200 with the breast pump 2. In some examples, the charging connector 200 may move along the preset direction relative to the fixing bracket 300 while rotating relative to the fixing bracket 300.

As shown in FIGS. 7 and 8, the charging connector 200 and the fixing bracket 300 are rotatably connected about a preset axis, and can move relatively along the preset direction, and the preset direction and an extending direction of the preset axis intersect each other.

For example, the direction from the first opening 301 toward the storage cavity 101 is defined as the preset direction, and the extending direction of the preset axis and the preset direction may be perpendicular to each other. For another example, the height direction D1 of the storage device 1 is defined as the preset direction, and the extending direction of the preset axis and the preset direction may be perpendicular to each other.

Thus, the charging connector 200 can have a plurality of motion forms relative to the fixing bracket 300, which increases a degree of freedom of the charging connector 200 relative to the fixing bracket 300, facilitates adjustment of the position of the charging connector 200, and thus facilitates the matching connection between the charging connector 200 and the breast pump 2.

As shown in FIGS. 5 to 8, one of the charging connector 200 and the fixing bracket 300 is provided with a rotating shaft 201 extending along the preset axis, and the other thereof is provided with a sliding groove 303 extending along the preset direction. The sliding groove 303 communicates with (e.g., is connected to) the accommodating space 302. The rotating shaft 201 is rotatably inserted into the sliding groove 303 so as to rotate relative to the fixing bracket 300 about the preset axis and slide relative to the fixing bracket 300 in the preset direction.

For example, the charging connector 200 is provided with a rotating shaft 201 extending along the preset axis, and the fixing bracket 300 is provided with a sliding groove 303 extending along the preset direction.

By rotatably inserting the rotating shaft 201 into the sliding groove 303, the sliding groove 303 can function as a limit guide on the rotating shaft 201, and the rotating shaft 201 can slide along the extending direction of the sliding groove 303, so that the charging connector 200 can rotate about the preset axis relative to the fixing bracket 300, and the charging connector 200 can slide along the preset direction relative to the fixing bracket 300.

In some examples, the extending direction of the sliding groove 303 is parallel to the height direction DI of the storage device 1. In other examples, the extending direction of the sliding groove 303 is parallel to the direction from the first opening 301 toward the storage cavity 101.

In some examples, the direction from the first opening 301 toward the storage cavity 101 is parallel to the height direction D1 of the storage device 1. In other examples, the direction from the first opening 301 toward the storage cavity 101 intersects the height direction D1 of the storage device 1.

The charging connector 200 and the corresponding fixing bracket 300 are slidably connected by two sliding grooves 303 and two rotating shafts 201, and the two rotating shafts 201 are respectively inserted into the two sliding grooves 303.

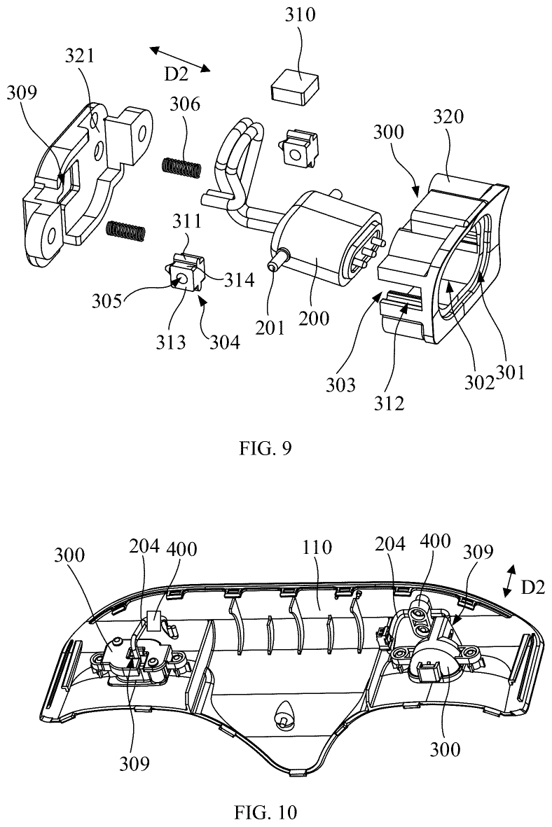

As shown in FIGS. 8 and 9, the storage device 1 comprises a slider 304. The slider 304 is slidably disposed in the sliding groove 303 so as to slide in the preset direction relative to the fixing bracket 300 in the sliding groove 303. The slider 304 is provided with a rotary hole 305, and the rotating shaft 201 is rotatably inserted into the rotary hole 305.

When a radial dimension of the rotating shaft 201 does not match a width dimension of the sliding groove 303, the slider 304 may be provided between the rotating shaft 201 and the sliding groove 303, and the slider 304 fits with the rotating shaft 201 and the sliding groove 303 separately, thereby improving stability of movement of the rotating shaft 201 in the sliding groove 303.

Specifically, the slider 304 may have two first side surfaces 313 and two second side surfaces 314. The two first side surfaces 313 disposed opposite along the axial direction of the rotary hole 305, and both ends of the rotary hole 305 are respectively opened on the two first side surfaces 313, so that the rotating shaft 201 at least passes through the first side surface 313. Further, one of the first side surfaces 313 of the slider 304 is disposed toward the accommodating space 302.

The two second side surfaces 314 are disposed opposite to two side walls of the sliding groove 303 in the width direction respectively. The two second side surfaces 314 may respectively be provided with limiting protrusions 311, and both side walls in the width direction of the sliding groove 303 may respectively be provided with limiting grooves 312, and an extending direction of the limiting groove 312 is parallel to the extending direction of the sliding groove 303. The limiting protrusion 311 is slidably inserted into the limiting groove 312 to limit the slider 304 from being disengaged from the limiting groove 312.

The storage device 1 comprises an elastic member 306 (e.g., a spring) that elastically abuts between the slider 304 and an end of the sliding groove 303 away from the first opening 301 and is configured to elastically support the slider 304 toward the first opening 301.

By elastically supporting the slider 304 in the direction toward the first opening 301, the elastic member 306 can support the charging connector 200 through the slider 304 and the rotating shaft 201, and the position of the charging connector 200 relative to the fixing bracket 300 can be kept stable, which is conducive to connection stability between the charging connector 200 and the breast pump 2. The elastic member 306 may be elastically deformed to allow the charging connector 200 to move relative to the fixing bracket 300.

The slider 304 may have a third side surface that intersects the first side surface 313 and the second side surface 314, and an end of the elastic member 306 may abut on the third side surface.

As shown in FIGS. 5 to 8, the first opening 301 faces the storage cavity 101 along a preset connection direction D2, and the charging connector 200 is configured to electrically contact the breast pump 2 in the preset connection direction D2. The charging connector 200 movably passes through the first opening 301 in the preset connection direction D2. This facilitates electrical connection between the charging connector 200 and the breast pump 2 and improves connection reliability therebetween.

As shown in FIGS. 5 to 8, the charging connector 200 is movable relative to the fixing bracket 300 along a height direction D3 of the breast pump 2, and the height direction D3 of the breast pump 2 is parallel to or intersects the preset connection direction D2.

Specifically, the breast pump 2 is provided with an electrical connection assembly, and the breast pump 2 is electrically connected with the charging connector 200 by the electrical connection assembly. The charging connector 200 is movable relative to the storage body 100 in the height direction D3 of the breast pump 2, so that the charging connector 200 can be matched and connected with the electrical connection assemblies of different heights when the storage device 1 stores the breast pump 2 having the electrical connection assemblies of different heights.

When the height direction D3 of the breast pump 2 is parallel to the preset connection direction D2, the electrical connection assembly may be provided at the bottom of the breast pump 2, and the charging connector 200 may be connected to the bottom of the breast pump 2. When the height direction D3 of the breast pump 2 intersects the preset connection direction D2, the electrical connection assembly may be provided on a side of the breast pump 2 toward the charging connector 200, and the charging connector 200 may be connected to the side of the breast pump 2.

As shown in FIG. 9, the fixing bracket 300 comprises a first housing 320 and a second housing 321 fixedly connected to enclose an accommodating space 302. For example, the first housing 320 and the second housing 321 may be fixedly connected by screws. Before the first housing 320 and the second housing 321 are fixedly connected, the charging connector 200 may be mounted into the accommodating space 302. This facilitates assembly of the charging connector 200 and is conducive to limiting the charging connector 200 by the fixing bracket 300.

As shown in FIG. 7, the fixing bracket 300 has a guide groove 307, and the guide groove 307 has two first inclined planes 308 disposed at an included angle and opposite to each other. The charging connector 200 has a guide portion 202 having two second inclined planes 203 that fit with the two first inclined planes 308. The first inclined planes 308 abut on the second inclined planes 203 respectively.

Since the guide groove 307 fits with the guide portion 202, when the charging connector 200 and the fixing bracket 300 are assembled, the guide groove 307 can guide the guide portion 202, which is conducive to assembly of the charging connector 200 and the fixing bracket 300 in place and improves an assembly effect.

Further, an opening of the guide groove 307 is provided toward the storage cavity 101 and communicates with the first opening 301.

The two sliding grooves 303 respectively penetrate side walls of the guide groove 307 where the two first inclined planes 308 are.

As shown in FIGS. 7, 8 and 10, the accommodating space 302 has a second opening 309. The charging connector 200 is connected to a wire 204, and the wire 204 extends from the inside of the accommodating space 302 to the outside of the accommodating space 302 through the second opening 309. The storage device 1 includes a wire limiter 400 mounted on the storage body 100 and positioned outside the accommodating space 302. The wire limiter 400 is configured to fix a part of the wire 204 extending out of the accommodating space 302.

The second opening 309 allows the wire 204 to enter the accommodating space 302 to supply power to the charging connector 200. When the charging connector 200 moves relative to the fixing bracket 300, the wire limiter 400 can limit the wire 204 outside the accommodating space 302, thereby reducing the risk of loosening and dislocation of the wire 204. For example, the wire limiter 400 may fix the wire 204 outside the accommodating space 302 to the storage body 100.

In some examples, the second opening 309 is disposed opposite the first opening 301. For example, the first opening 301 is provided in the first housing 320, and the second opening 309 is provided in the second housing 321.

In other examples, a direction of the first opening 301 toward the inside of the accommodating space 302 intersects a direction of the second opening 309 toward the inside of the accommodating space 302. For example, the second opening 309 is provided in the guide groove 307, the wire 204 may pass through the guide groove 307 to be connected to the charging connector 200, and the guide portion 202 is formed on the outer periphery of a part of the wire 204 in the guide groove 307.

As shown in FIGS. 5 to 8, the charging connector 200 is provided with a first power supply contact 205 and a first alignment magnet 206, and the breast pump 2 is provided with a second power supply contact 21 and a second alignment magnet 22. The first power supply contact 205 is electrically connected with the second power supply contact 21 so that the charging connector 200 is electrically connected with the breast pump 2. The first alignment magnet 206 is magnetically connected with the second alignment magnet 22 so that the first power supply contact 205 is electrically connected with the second power supply contact 21.

The magnetic connection between the first alignment magnet 206 and the second alignment magnet 22 is conducive to automatic and accurate alignment of the first power supply contact 205 with the second power supply contact 21. Specifically, when the breast pump 2 needs to be charged, after the breast pump 2 is placed in the storage cavity 101, the second alignment magnet 22 can attract the first alignment magnet 206 to drive the charging connector 200 to move, so that the first power supply contact 205 and the second power supply contact 21 approach and contact each other, thereby realizing automatic alignment electrical connection between the charging connector 200 and the breast pump 2.

Further, the first alignment magnet 206 may be disposed on the periphery of the first power supply contact 205, and the second alignment magnet 22 may be disposed on the periphery of the second power supply contact 21.

The first power supply contact 205 includes two high potential contacts and a ground contact, the two high potential contacts are symmetrically disposed on both sides of the ground contact, and the second power supply contact 21 only needs to be connected to the ground contact and one of the high potential contacts. In this way, assembly fault tolerance of the charging connector 200 can be improved.

The second power supply contact 21 and the second alignment magnet 22 may be provided on the main engine 20, so that the main engine 20 can be detached from the breast pump 2 and then placed in the storage cavity 101 for charging.

As shown in FIGS. 5 to 8, the charging connector 200 is provided with a first limit magnet 207, and the fixing bracket 300 is provided with a second limit magnet 310. The first limit magnet 207 is magnetically connected with the second limit magnet 310 to limit movement of the charging connector 200 relative to the fixing bracket 300.

When the charging connector 200 is in an idle state, the first limiting magnet 207 and the second limiting magnet 310 are magnetically connected, so that the charging connector 200 and the fixing bracket 300 can be kept relatively fixed, facilitating storing the charging connector 200. After charging of the breast pump 2 ends, the second limit magnet 310 can drive the charging connector 200 to reset in the idle state by attracting the first limit magnet 207.

Further, the charging connector 200 is provided with a first limit magnet 207 and a first alignment magnet 206, the fixing bracket 300 is provided with a second limit magnet 310, and the breast pump 2 is provided with a second alignment magnet 22. A magnetic attraction force between the first alignment magnet 206 and the second alignment magnet 22 may be greater than a magnetic attraction force between the first limit magnet 207 and the second limit magnet 310. When the breast pump 2 needs to be charged, after the breast pump 2 is placed in the storage cavity 101, the second alignment magnet 22 can attract the first alignment magnet 206 to drive the charging connector 200 to move, and the first limit magnet 207 and the second limit magnet 310 are separated from each other.

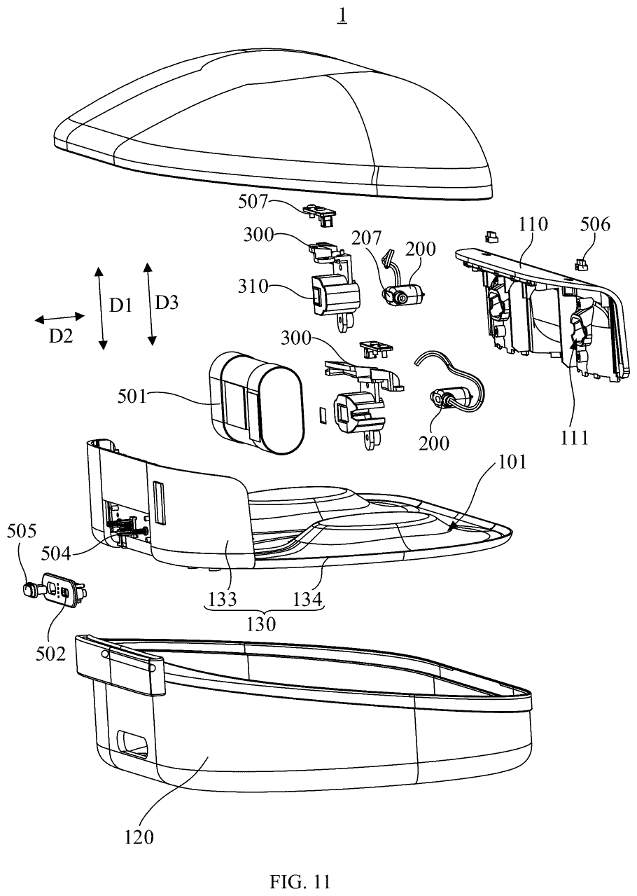

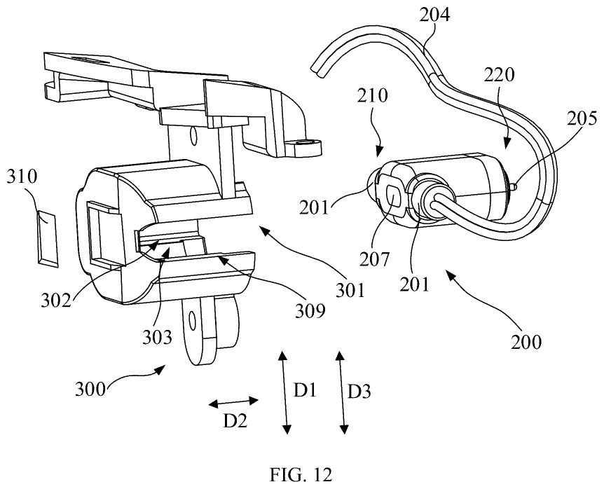

As shown in FIGS. 11 and 12, the fixing bracket 300 is provided with two sliding grooves 303, and a bottom wall of one of the sliding grooves 303 is provided with a second opening 309 communicating with the accommodating space 302. The charging connector 200 is provided with two rotating shafts 201 respectively rotatably inserted into the two sliding grooves 303. The charging connector 200 is connected with a wire 204 passing through one of the two rotating shafts 201 and extending out of the accommodating space 302 through the second opening 309. Thus, arrangement of the wire 204 and the rotating shaft 201 can be made more compact, which can save the space occupied by the wire 204 and the rotating shaft 201 on the fixing bracket 300, and is conducive to disposing other parts on the fixing bracket 300.

Further, an end of the charging connector 200 facing away from the storage cavity 101 is provided with a first limit magnet 207, a side of the fixing bracket 300 facing the storage cavity 101 is provided with a second limit magnet 310, and the first limit magnet 207 and the second limit magnet 310 are configured to attract each other, so that the charging connector 200 and the fixing bracket 300 are kept relatively fixed.

As shown in FIGS. 3, 11 and 12, the storage body 100 is provided with a partition member 110 and an accommodating cavity 102, and the storage cavity 101 and the accommodating cavity 102 are respectively located on two sides of the partition member 110. The partition member 110 is provided with a mounting hole 111 that connects the storage cavity 101 with the accommodating cavity 102, and the fixing bracket 300 is mounted on a side of the partition member 110 facing the accommodating cavity 102 and is provided opposite to the mounting hole 111. At least part of the charging connector 200 is movably stopped between the partition member 110 and the fixing bracket 300 and is exposed to the storage cavity 101 through the mounting hole 111.

Thus, the charging connector 200 can be stored outside the storage cavity 101, thereby reducing interference of the charging connector 200 on the storage of the storage cavity 101.

In some examples, the charging connector 200 may be completely positioned between the partition member 110 and the fixing bracket 300 when stored. In other examples, the charging connector 200 may be partially positioned between the partition member 110 and the fixing bracket 300 when stored, and a part of the charging connector 200 may be positioned within the mounting hole 111.

In some examples, the second power supply contact 21 of the breast pump 2 may be connected to the charging connector 200 through the mounting hole 111.

As shown in FIGS. 3, 11 and 12, the storage device 1 includes a power supply assembly 500 provided in the accommodating cavity 102. The charging connector 200 has an electrical receiving end 210 and an electrical output end 220 connected to each other. The electrical receiving end 210 is movably stopped between the partition member 110 and the fixing bracket 300 and is electrically connected with the power supply assembly 500. The electrical output end 220 is configured to move into the storage cavity 101 through the mounting hole 111 to be electrically connected with the breast pump 2, and the electrical output end 220 is also configured to leave the storage cavity 101 through the mounting hole 111 to be stored. Thus, the charging connector 200 is not separated from the fixing bracket 300, which improves movement stability of the charging connector 200, facilitates storage of the charging connector 200 and improves the aesthetic degree.

Further, the rotating shaft 201 is located at the electrical receiving end 210, and an end of the sliding groove 303 close to the storage cavity 101 extends to the partition member 110, so that the rotating shaft 201 is movably stopped between the partition member 110 and the fixing bracket 300.

As shown in FIG. 3 and FIG. 11, a size of an end of the mounting hole 111 close to the storage cavity 101 is larger than a size of an end of the mounting hole 111 close to the accommodating cavity 102. Thus, the size of the end of the mounting hole 111 close to the accommodating cavity 102 may be smaller and can fit with the size of the charging connector 200, so that the charging connector 200 is kept fixed when stored; and the size of the end of the mounting hole 111 close to the storage cavity 101 may be larger, so that the position and posture of the charging connector 200 can be adjusted more freely by rotation and sliding when the charging connector 200 is electrically connected with the breast pump 2, facilitating the connection and alignment of the charging connector 200 with the breast pump 2.

As shown in FIG. 11, a light-emitting unit 507 is mounted on the fixing bracket 300 and electrically connected with the charging connector 200, and a light guide member 506 can penetrate the partition member 110. The light-emitting unit 507 is configured to emit light when the charging connector 200 is electrically connected with the breast pump 2, and the light guide member 506 is configured to conduct the light emitted by the light-emitting unit 507 to prompt the user.

As shown in FIGS. 1, 2, and 10, the storage device 1 includes a power supply assembly 500, the storage body 100 is provided with the accommodating cavity 102 disposed at an interval from the storage cavity 101, and the power supply assembly 500 is disposed in the accommodating cavity 102 and electrically connected with the charging connector 200. For example, the wire 204 may extend from the accommodating cavity 102 to the accommodating space 302, and the power supply assembly 500 is electrically connected with the charging connector 200 through the wire 204. The accommodating cavity 102 facilitates assembly of the power supply assembly 500, and the storage body 100 can protect the power supply assembly 500.

As shown in FIGS. 1, 2, and 10, the storage body 100 comprises a connection housing 120, the partition member 110 and a base 130. The base 130 is connected to the connection housing 120 and the partition member 110 separately. The connection housing 120, the partition member 110, and the base 130 enclose the storage cavity 101. The base 130 and the partition member 110 enclose the accommodating cavity 102. Further, the partition member 110 and the base 130 are disposed inside the connection housing 120, and partition a space inside the connection housing 120 into the storage cavity 101 and the accommodating cavity 102. Thus, the storage cavity 101 and the accommodating cavity 102 can be disposed at intervals, facilitating assembly of the storage device 1.

The connection housing 120 may be made of a braided fabric material and provided with a zipper 121, so that the storage body 100 can be partially separated and opened to facilitate taking and placing the breast pump 2. The base 130 may be made of a plastic material having a supporting and shaping function.

The base 130 may be configured to support the breast pump 2. Further, the base 130 may be provided with a circular protrusion 131, and after the breast pump 2 is placed into the storage cavity 101, the outer cover assembly 10 may face the circular protrusion 131, so that the circular protrusion 131 can position the outer cover assembly 10.

The base 130 and the partition member 110 may be connected by a threaded connecting member. The base 130 comprises an upper support portion 133 and a bottom support portion 134, and the bottom support portion 134 is configured to support the breast pump 2. The upper support portion 133 is provided on a side of the bottom support portion 134 facing the breast pump 2, and the upper support portion 133 is configured to support the connection housing 120. The partition member 110 may be disposed opposite the upper support portion 133 and form a sidewall of the accommodating cavity 102. A part of the bottom support portion 134 may form a bottom wall of the accommodating cavity 102.

The partition member 110 may be positioned between the accommodating cavity 102 and the storage cavity 101. The partition member 110 may be provided with a mounting hole 111 that connects the storage cavity 101 with the accommodating cavity 102, and the fixing bracket 300 may be mounted on a side of the partition member 110 facing the accommodating cavity 102 so that the first opening 301 is opposite to the mounting hole 111, and the charging connector 200 may extend from the accommodating space 302 to the storage cavity 101 through the first opening 301 and the mounting hole 111.

Further, the wire 204 outside the accommodating space 302 may be fixed on the partition member 110 by the wire limiter 400.

The storage body 100 may be further provided with a handle 122 to facilitate carry of the storage device 1 by a user. A support feet 132 are provided at the bottom of the storage device 1.

As shown in FIGS. 1 and 2, the power supply assembly 500 includes an energy storage element 501 (e.g., battery) electrically connected with the charging connector 200 to supply power to the breast pump 2 through the charging connector 200. By providing the energy storage element 501, the storage device 1 can charge the breast pump 2 without being connected to an external power supply.

As shown in FIGS. 1 and 2, the storage body 100 may be provided with a charging interface 502, through which the energy storage element 501 is connected with an external power supply. By providing the charging interface 502, the external power supply can be connected to the charging interface 502, thereby supplying power to the energy storage element 501 and the breast pump 2. Further, the charging interface 502 is provided toward the outside of the storage body 100, and the storage body 100 may be provided with an opening to avoid the charging interface 502, facilitating connection with the external power supply. The charging interface 502 may be a micro USB interface, a type-C interface, or a lighting interface.

As shown in FIGS. 1 and 2, the power supply assembly 500 includes a detection circuit board 503 electrically connected with the energy storage element 501 and configured to obtain power amount information of the energy storage element 501. The detection circuit board 503 detects a remaining amount of power of the energy storage element 501 to manage the charging function of the storage device 1.

Further, the power supply assembly 500 further includes a prompt member 504 and a trigger member 505, the prompt member 504 is electrically connected with the detection circuit board 503 and the trigger member 505 separately and is configured to send prompt information indicating the power amount (e.g., battery percentage) of the energy storage element 501, and the trigger member 505 is touched by a user to trigger the prompt member 504 to send the prompt information. For example, the prompt member 504 is a light indicating information associated with the power consumption of energy storage element 501, and the trigger member 505 is a button or a slide key.

The prompt member 504 and the trigger member 505 are provided toward the outside of the storage body 100, and the storage body 100 may be provided with an opening to avoid the prompt member 504 and the trigger member 505, so that the prompt member 504 and the trigger member 505 are exposed to the outside world, facilitating touching the trigger member 505 and observing the indicating light by the user.

As shown in FIGS. 1 and 2, the number of the charging connectors 200 may be at least two, and at least two breast pumps 2 (e.g., a pair of breast pumps) are accommodated in the storage cavity 101. The at least two charging connectors 200 are electrically connected to the at least two breast pumps 2 respectively.

For example, the number of the charging connectors 200 is two, and the two breast pumps 2 are accommodated in the storage cavity 101. The two charging connectors 200 are electrically connected to the two breast pumps 2 respectively. The two charging connectors 200 may be the same or different. Thus, the storage device 1 can meet charging requirements and storage requirements of the at least two breast pumps 2.

In summary, the present examples can realize that by setting the charging connector 200 to be movable relative to the storage body 100 to adjust the position of the charging connector 200. The charging connector 200 can be matched and connected with different breast pumps 2 when the storage device 1 stores breast pumps 2 of different structures and sizes, thereby improving the compatibility of the storage device 1 with the breast pumps 2 of different structures and sizes during charging, so that the storage device 1 has a wider range of application scenarios.

The above are only some examples of the present disclosure and do not limit the scope of the disclosure. Any equivalent structure or process transformation made using the contents of description and drawings of the present disclosure, or direct or indirect use thereof in other related technical fields should be included in the scope of the present disclosure.

What is claimed is:

1. A storage device of a breast pump, comprising:
a storage body comprising a storage cavity;
a charging connector, wherein the charging connector is movable relative to the storage body to adjust a position of the charging connector, and the charging connection is electrically connected to the breast pump based on a position of the breast pump in the storage cavity; and
a fixing bracket disposed on the storage body and movably connected to the charging connector.

2. The storage device according to claim 1, wherein:
the fixing bracket comprises an accommodating space having a first opening, and
at least part of the charging connector is in the accommodating space, is configured to pass through the first opening, and is exposed relative to the storage cavity through the first opening.

3. The storage device according to claim 1, wherein the charging connector and the fixing bracket are rotatably connected.

4. The storage device according to claim 1, wherein the charging connector and the fixing bracket are relatively movable along a direction to adjust the position of the charging connector.

5. The storage device according to claim 4, wherein:
the charging connector and the fixing bracket are rotatably connected about an axis and are relatively movable along the direction, the direction and an extending direction of the axis intersecting each other.

6. The storage device according to claim 5, wherein:
one of the charging connector and the fixing bracket comprises a rotating shaft extending along the axis,
the other one of the charging connector and the fixing bracket comprises a sliding groove extending along the direction, and
the rotating shaft is rotatably inserted into the sliding groove so as to rotate relative to the fixing bracket about the axis and slide relative to the fixing bracket along the direction.

7. The storage device according to claim 6, wherein:
the fixing bracket comprises two sliding grooves,
a bottom wall of one of the two sliding grooves comprises a second opening,
the charging connector comprises two rotating shafts respectively rotatably inserted into the two sliding grooves, and
the charging connector is connected to a wire passing through one of the two rotating shafts and extending through the second opening.

8. The storage device according to claim 6, further comprising:
a slider slidably disposed in the sliding groove so as to slide along the direction relative to the fixing bracket in the sliding groove, and
the slider comprises a rotary hole into which the rotating shaft is rotatably inserted.

9. The storage device according to claim 8, further comprising:
a spring configured to elastically abut between the slider and an end of the sliding groove.

10. The storage device according to claim 2, wherein:
the fixing bracket comprises a guide groove having two first inclined planes opposite to each other,
the charging connector has a guide portion having two second inclined planes that fit with the two first inclined planes, and each of the two first inclined planes abuts on a respective one of the second inclined planes.

11. The storage device according to claim 2, wherein:
the accommodating space has a second opening,
the charging connector is connected to a wire passing through the second opening and extending out of the accommodating space through the second opening,
the storage device further comprises a wire limiter mounted on the storage body and positioned outside the accommodating space, and
the wire limiter is configured to fix a part of the wire extending out of the accommodating space.

12. The storage device according to claim 1, wherein:
the charging connector comprises a first power supply contact and a first alignment magnet,
the breast pump comprises a second power supply contact and a second alignment magnet,
the first power supply contact is electrically connected with the second power supply contact, and
the first alignment magnet is magnetically coupled to the second alignment magnet.

13. The storage device according to claim 1, wherein:
the charging connector comprises a first magnet,
the fixing bracket comprises a second magnet, and
the first magnet is magnetically coupled to the second magnet to limit movement of the charging connector relative to the fixing bracket.

14. The storage device according to claim 1, wherein:
the storage body comprises a partition and an accommodating cavity,
the storage cavity and the accommodating cavity are respectively located on two sides of the partition,
the partition comprises a mounting hole that connects the storage cavity with the accommodating cavity,
the fixing bracket is mounted on a side of the partition facing the accommodating cavity and is disposed opposite to the mounting hole, and
at least part of the charging connector is between the partition and the fixing bracket and is exposed to the storage cavity through the mounting hole.

15. The storage device according to claim 14, comprising:
a power supply assembly disposed in the accommodating cavity,
the charging connector has an electrical receiving end and an electrical output end,
the electrical receiving end is limited between the partition and the fixing bracket, and is electrically connected to the power supply assembly, and
the electrical output end is configured to move into the storage cavity through the mounting hole to be electrically connected to the breast pump, and is configured to leave the storage cavity through the mounting hole.

16. The storage device according to claim 15, wherein:
the power supply assembly comprises a battery electrically connected with the charging connector to supply power to the breast pump through the charging connector,
the storage body comprises a charging interface, through which the battery is connected with an external power supply.

17. The storage device according to claim 1, wherein:
a number of the charging connectors is at least two, and at least two of the breast pumps are accommodated in the storage cavity, and
at least two of the charging connectors are electrically connected to at least two of the breast pumps respectively.

18. An apparatus comprising:
a breast pump; and
a storage device comprising:
a storage body comprising a storage cavity;
a charging connector, wherein the charging connector is movable relative to the storage body to adjust a position of the charging connector, and the charging connection is electrically connected to the breast pump based on a position of the breast pump in the storage cavity; and
a fixing bracket disposed on the storage body and movably connected to the charging connector.

19. The apparatus according to claim 18, wherein:
the fixing bracket comprises an accommodating space having an opening, and
at least part of the charging connector is in the accommodating space, is configured to pass through the opening, and is exposed relative to the storage cavity through the opening.

20. A storage device for a breast pump comprising:
a storage body comprising a storage cavity;
a charging connector electrically connected with the breast pump; and
a bracket disposed on the storage body, wherein the bracket and the charging connector are movably connected so that the charging connector is movable relative to the storage body.
